(19) United States
(12) Patent Application Publication
(10) Pub. No.: US 2026/0090919 A1
(43) Pub. Date: Apr. 2, 2026

(54) NASAL COMPRESSION AND/OR DRUG DELIVERY DEVICE

(71) Applicant: Nasaclip Inc., Baltimore, MD (US)

(72) Inventors: Elizabeth P. Clayborne, Baltimore, MD (US); Jordan D. Hayes, Baltimore, MD (US); Soren B. Sibbesen, Bel Air, MD (US)

(73) Assignee: Nasaclip Inc., Baltimore, MD (US)

(21) Appl. No.: 18/900,023

(22) Filed: Sep. 27, 2024

(51) Int. Cl. A61F 13/20; A61B 17/12; A61B 17/122; A61B 17/24

(52) U.S. Cl. CPC A61F 13/2005; A61B 17/122; A61B 17/24; A61F 13/2074; A61B 2017/12004

(57) ABSTRACT

A nose compression device stops nosebleeds and is a medication applicator. The device has two L-shaped support members, each having elongated arms with a compression pad formed at a distal end, and an elongated engagement member at the proximal end. The engagement members slidably couple with one another and couple with sponge(s) that are inserted into a patient's nose. To apply the device, the engagement members are moved inward until the compression pads press against a user's nose. The outward force created by the user's nose pressing outward against the compression pads, creates a radial force at the engagement members that allows the device to be further pressed inward, but prevents the engagement members from being moving inward, thereby locking the device to the user's nose. A releasing outward force can be applied along the engagement members to release the radial force and move the engagement members outward so the device can be removed from the user's nose. Releasable locking features are formed on the engagement members to prevent the engagement members from inadvertently coming apart. However, a user can unlock the locking features to remove and replace or reuse the sponge(s).

### BACKGROUND

Sixty percent of people will experience a nosebleed in their lifetime. In the United States, nosebleeds account for approximately 1 in 200 emergency department visits, which is over 500,000 visits to the emergency room in the United States annually of which nearly 90% are safely sent home. Nosebleeds are very messy, bloody, anxiety provoking experiences that could be easily managed at home if treated appropriately. Epistaxis, the medical term for nosebleed, is one of the most common ear, nose, and throat emergencies. Epistaxis has a bimodal age distribution, with most cases in children 2-10 years old and adults 50-80 years old. Certain high-risk groups, such as the elderly, require rapid intervention to stem bleeding and prevent further complications.

Nosebleeds are commonly mismanaged when they first begin. One common mistake in treating nosebleeds relates to improper compression. Nosebleeds should be treated by applying appropriate pressure to the soft side walls of the nose, for 10 to 20 minutes without interruption, and positioning the head slightly forward. While this sounds simple, it is hard to do. Another common mistake is inadequate compression time. Nosebleeds need compression for 10 to 20 minutes without interruption. Due to arm fatigue, these steps are hard to do consistently.

Medications can be used to constrict vessels to help stop bleeding. However, there is little education or awareness of how, when and what medications can be used. At home, children and elderly may easily tire, forget, or not understand these steps for successful nosebleed rescue.

There is a growing demand for efficient, non-invasive medication administration. Current methods range from atomizer sprays and aerosols to oral and injectable routes. However, those often fall short due to their limited mucosal retention and inconsistent administration techniques, which undermines the drug effectiveness, especially for children due to anatomical considerations and the need for non-invasive options.

### SUMMARY

Accordingly, it is one object of the present disclosure to provide a device that treats nosebleeds. It is another object of the disclosure to provide a device that treats nosebleeds by applying a compression force against the lateral side surfaces of the user's nose. It is another object of the disclosure to provide a device that treats nosebleeds by inserting a sponge, absorbent material or synthetic inside the user's nasal passage. It is another object of the disclosure to provide a device that treats nosebleeds by applying a compression force against the lateral side surfaces of the user's nose at the same time it inserts a sponge inside the user's nasal passage. It is a further object of the disclosure to provide a nose compression device that can be used hands-free and that does not obstruct the user's mouth and/or eyes and can be used with a surgical mask.

The disclosure describes a device for drug delivery through innovative pre-medicated sponges for both local and systemic effects. This device targets a diverse customer base, including emergency medicine physicians seeking rapid systemic drug administration alternatives and patients, particularly within the pediatric population, in need of a safe and user-friendly method for self-administering emergency medications. The device maximizes drug absorption through consistent nasal pressure, a method far superior to existing practices in terms of efficiency and patient compliance. The device's versatility extends to enhancing the delivery of a range of medications, from hemostatic agents for acute hemorrhagic conditions to antidotes and antibiotics for emergency care, as well as analgesics and anti-anxiety medications. This broad utility can become an essential component in both emergency medical kits and household medicine cabinets, in addition to traditional healthcare settings.

The intranasal drug delivery device addresses both pharmacologic dynamics and a user-centric design. This device taps into the intranasal route's unique advantage of rich vascularization, offering direct access for rapid medication action, comparable to IV administrations. The device exerts consistent pressure on crucial nasal absorption areas, enhancing drug retention for poorly absorbed medications, and accelerating systemic absorption, thereby boosting bioavailability.

The design significantly streamlines the drug administration process, making it more user-friendly compared to conventional nasal devices that often come with complicated instructions. By eliminating the need for manual nasal compression, the device ensures a simpler, more efficient delivery method, which is particularly valuable in urgent care scenarios where swift and reliable medication administration is paramount. This ease of use, coupled with the device's ability to improve drug efficacy, resonates strongly with healthcare professionals and patients alike, underscoring a pressing market demand for more effective intranasal drug delivery solutions.

The device is particularly advantageous for patients requiring immediate medication, offering a user-friendly alternative that can be lifesaving in instances of seizure clusters, opioid overdoses, or severe allergic reactions. Additionally, its design to apply medicated pressure directly addresses the immediate needs of those suffering from acute nosebleeds, often encountered in sports or accidental injuries, allowing for rapid on-field treatment and the possibility for athletes to continue play without interruption.

Moreover, the device can be used at rural and underserved communities, older adults, and uninsured families, who frequently lack access to quality clinical care and are compelled to rely on inadequate home remedies or face costly emergency room visits. The device's simplicity, effectiveness, and home usability present an opportunity to bridge the healthcare gap in these communities, providing a reliable care solution that mitigates the need for expensive and time-consuming hospital care.

In addition, the design is fully plastic, ensuring that athletes can safely return to play with the device in place, further underscoring its versatility and user-centric design. This strategic adaptation broadens its applicability across various emergency scenarios.

These and other objects of the disclosure, as well as many of the intended advantages thereof, will become more readily apparent when reference is made to the following description, taken in conjunction with the accompanying drawings. This summary is not intended to identify all essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1A is a perspective view of a nasal compression and/or drug delivery device in accordance with an embodiment of the disclosure;

FIG. 1B is a front view of the device of FIG. 1A, in a partly-opened position;

FIG. 1C is a side view of the device of FIG. 1A;

FIG. 1D is a front view of the device of FIG. 1A, in a fully closed position;

FIG. 1E is a perspective view of the device of FIG. 1A;

FIG. 1F is a front view of the device of FIG. 1A;

FIG. 1G is a rear view of the device of FIG. 1A;

FIG. 1H is a top view of the device of FIG. 1A;

FIG. 1I is a bottom view of the device of FIG. 1A;

FIG. 1J is a side view of the device of FIG. 1A;

FIG. 2A is a detailed perspective view of a first male external pressure member having a male support engagement member;

FIG. 2B is a cross-section view of the first male external pressure member of FIG. 2A, without the compression head;

FIG. 2C is a top view of the first male external pressure member of FIG. 2A;

FIG. 3A is a detailed perspective view of a second female external pressure member having a female support engagement member;

FIG. 3B is a cross-section view of the second female external pressure member of FIG. 3A, without the compression head;

FIG. 3C is a top view of the second female external pressure member of FIG. 3A;

FIG. 4A is a top view of the first male external pressure member received in the second female external pressure member, and in a fully expanded position;

FIG. 4B is a side view of the first male external pressure member received in the second female external pressure member, and in a fully expanded position;

FIG. 4C is a top view of the first male external pressure member received in the second female external pressure member, and in a fully retracted position;

FIG. 5A is a perspective top view of an internal member in an unfolded storage position;

FIG. 5B is a rear perspective view of the internal member of FIG. 4A, in a folded assembled position;

FIG. 6A is a side view of the fully assembled device showing movement of the male and female external pressure member between a pre-insertion position and an insertion position;

FIG. 6B is a front view of the fully assembled device in a pre-insertion position;

FIG. 6C is a front view of the fully assembled device in an insertion position;

FIG. 7A is a front view of the device in a pre-insertion position and aligned with the nasal passages of a user;

FIG. 7B is a perspective view of the device in a pre-insertion position and aligned with the nasal passages of a user;

FIG. 7C is a side view of the device showing the arms pivoting from the pre-insertion position to a post-insertion position to align the pinch pads with the outer sides of the user's nose;

FIG. 7D is front view of the device with the arms in the outward post-insertion and pre-grip position with the pinch pads aligned with the outer sides of the user's nose;

FIG. 7E is a perspective view of the device with the arms at an inward grip position so the pinch pads grip the outer surface of the user's nose; and

FIG. 7F is a front view of the device with the arms at an inward grip position so the pinch pads grip the outer surface of the user's nose;

The figures show one illustrative embodiment of the present disclosure. Other embodiments can have components of different scale. Like numbers used in the figures may be used to refer to like components. However, the use of a number to refer to a component or step in a given figure has a same structure or function when used in another figure labeled with the same number, except as otherwise noted.

### DETAILED DESCRIPTION

In describing the illustrative, non-limiting embodiments of the disclosure illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments of the disclosure are described for illustrative purposes, it being understood that the disclosure may be embodied in other forms not specifically shown in the drawings.

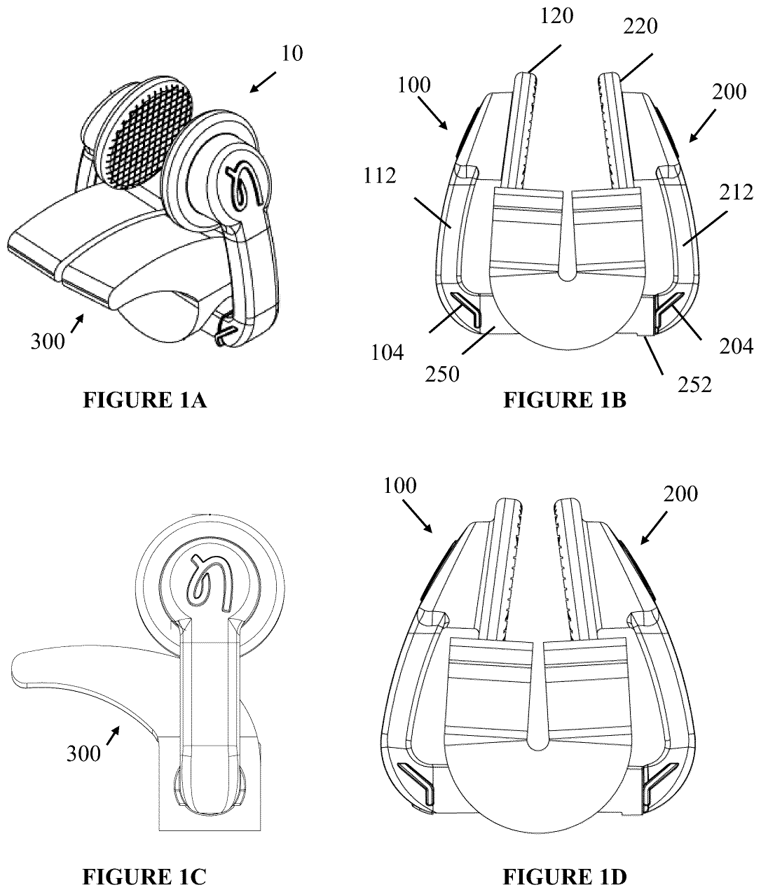

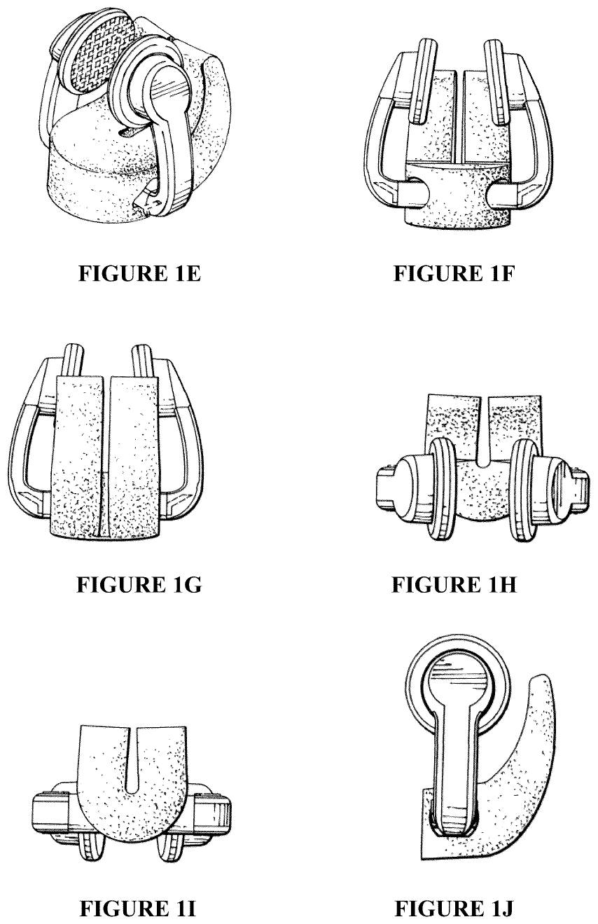

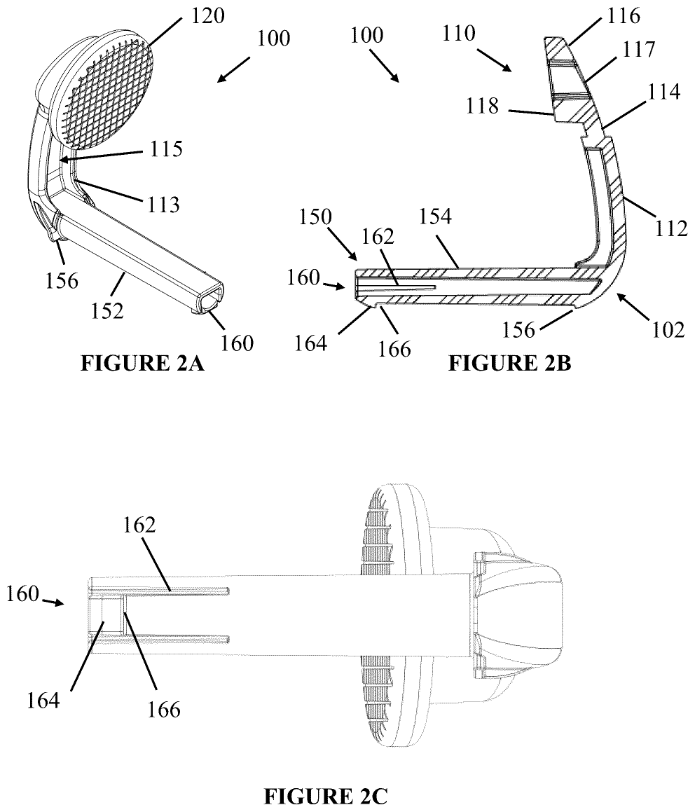

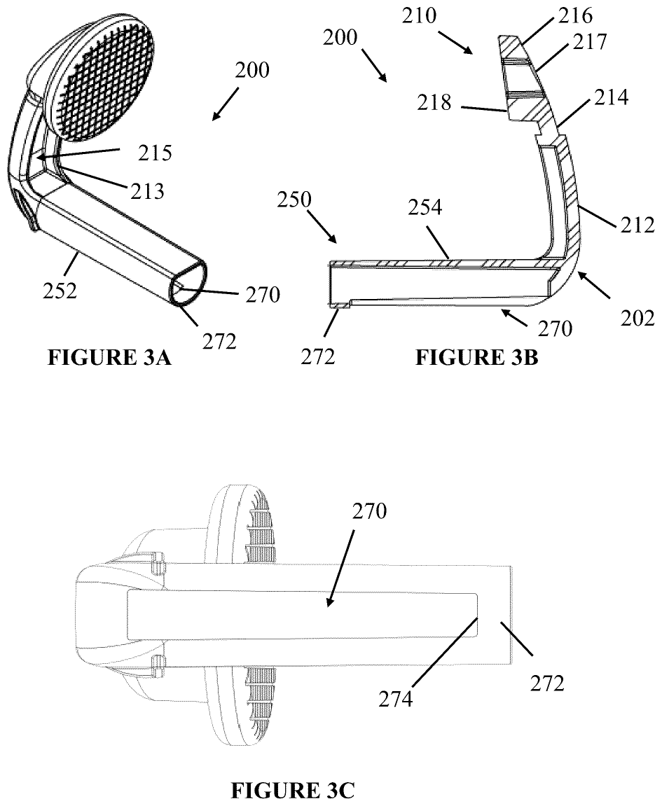

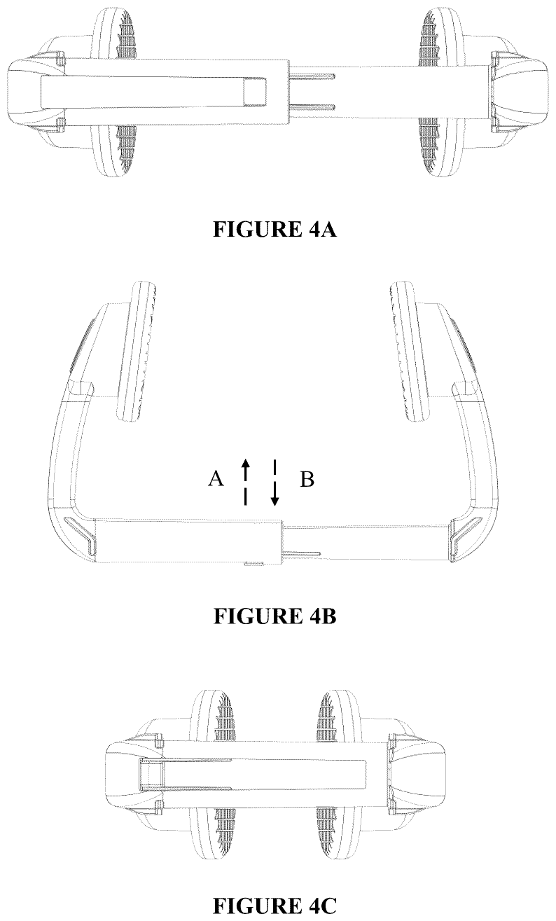

Turning to the drawings, FIGS. 1A-1J show a nasal compression and/or drug delivery device 10 in accordance with one non-limiting illustrative example embodiment of the disclosure. The device 10 is designed to treat anterior epistaxis or nosebleeds. The compression device 10 has a first external pressure member 100 (FIGS. 2A, 2B), a second external pressure member 200 (FIGS. 3A, 3B), and an internal member 300 (FIGS. 4A, 4B).

The external pressure members 100, 200 and provide an inward pressure against the outer surface of the nasal passage to pinch the nasal passage closed and seal any broken nasal blood vessel. At least a portion of the internal member 300 may contact the inner surface of the nasal passage and any broken nasal blood vessel to further seal any broken nasal blood vessel. In some embodiments, the internal member 300 may release liquid medication when the device is squeezed shut and facilitate distribution over nasal mucosa where bleeding originates (e.g., Kiesselbach's plexus).

### First Male External Pressure Member 100 (FIGS. 2A, 2B, 2C)

Referring to FIGS. 1B, 2A, 2B, 2C, the first male external pressure member 100 is shown. The male external pressure member 100 has a first elongated side arm 110, and a first elongated male engagement member 150. The first side arm 110 is formed integral with the first male engagement member 150.

The first side arm 110 is elongated and extends along a first arm longitudinal axis, with a distal end and a proximal end. A first curved bend, elbow or corner 102 is formed at the proximal end of the first side arm 110. The first side arm 110 includes a first elongated main body section 112, a first neck 114, and a first head 116. The head 116 is formed at the distal end of the first side arm 110, and has a first through-hole 117 that extends completely through the entire width of the head 116, transverse to the first longitudinal axis. The through-hole 117 can be a circle or oval shape, with an added notch at the top and/or bottom of the circle, to resemble a keyhole shape.

The head 116 also has a circular inward-facing face or surface 118. A first neck 114 is formed between the first main body section 112 and the first head 116. The first neck 114 is smaller than the main body section 112 to form a step-in or reduced portion that allows for material thickness of the pinch pad 120 silicone to be molded or placed over and about the head 116 and neck 114 (compare FIGS. 2A, 2B), so that the pinch pad 120 is flush with the inner and outer surfaces of the main section 112 at the neck 114. In addition, a rib can extend longitudinally at the reduced portion of the neck 114 from the main body section 112 to the head 116. The rib extends outward from the reduced portion to prevent rotation of the silicone. The main body section 112 extends from the curved corner 102 at the proximal end of the first side arm 110, to the neck 114.

As best shown in FIG. 2B, the main body section 112 has a rectangular cross-section. A channel 115 is formed at the inside of the main body section 112, which in turn forms side rails 113 that extend longitudinally along the forward-edge and rear-edge of the main body section 112, and to the rounded corner 102. The side rails 113 provide added support and strength to the main body section 112 and the rounded corner 102.

A first compression or pinch pad 120 is formed separately over and about the head 116, in the through-hole 117 and about the neck 114, to be coupled therewith. The through-hole 117 (including the notches), neck 114 and neck rib cooperate to enhance the coupling between the pinch pad 120 and the head 116 and neck 114, and to prevent rotation of the pinch pad 120 with respect to the first male external pressure member 100. Other coupling features can be added to further enhance coupling and prevent rotation of the pinch pad 120 with respect to the male external pressure member 100. The pinch pad 120 has a larger diameter than the first head 116, and has an inner surface that is cross-hatched to better grip the user's nose.

The first male engagement member 150 is a linear elongated tube. In some embodiments, the tube has an irregular cross-sectional shape, for example, it can have symmetry in a radial direction, but lacks rotational symmetry, or is rotationally asymmetric. In the embodiment shown, the engagement member 150 has a rounded lower portion 152 and a flat top portion 154. The rounded lower portion 152 can be a circular arc and the top portion 154 is a chord connecting the endpoints of the lower portion 152, to resemble a U-shape with a top that is curved inward.

In the embodiment shown, the male engagement member 150 has a first engagement longitudinal axis, a distal end, and a proximal end. The male engagement member 150 is slightly smaller than the curved corner 102 (which is the same size as the first main body section 112), and a portion of the proximal end of the male engagement member 150 is received by the channel 115 of the curved corner 102 forming a lip 156 inward from the extreme proximal end of the lower portion 152 of the male engagement member 150, whereby the lip 156 is between the curved corner 102 and the male engagement member 150. The male engagement member 150 is hollow, with a central opening extending the entire length of the engagement member 150.

As best shown in FIG. 2C, a sliding adjustment and lock mating feature in the form of an elongated tongue 160 is formed at the distal end of the male engagement member 150. Two parallel elongated slots 162 are formed to extend completely through the lower portion 152, at the two longitudinal sides of the tongue 160. The slots 162 extend from the extreme distal end of the male engagement member 150 and partially longitudinally inward, as shown. The slots 162 define the tongue 160, and enable the tongue to flex inward and outward, and being biased to the relaxed position where it is straight with the bottom of the lower portion 152. A locking tab 164 is formed at the distal end of the tongue 160, at the bottom of the engagement member 150. The tab 164 forms a ramp that is sloped outward as it extends from the distal end of the tongue 160 inward, forming a locking lip 166 between the tab 164 and the bottom outer surface of the lower portion 152 of the engagement member 150.

### Second Female External Pressure Member 200 (FIGS. 3A, 3B, 3C)

Referring to FIGS. 1B, 3A, 3B, 3C, the second female external pressure member 200 is shown. The female external pressure member 200 has a second elongated side arm 210, and a second elongated male engagement member 250. The second side arm 210 is formed integral with the second male engagement member 250.

The second side arm 210 is elongated and extends along a second arm longitudinal axis, with a distal end and a proximal end. A second curved bend, elbow or corner 202 is formed at the proximal end of the second side arm 210. The second side arm 210 includes a second elongated main body section 212, a second neck 214, and a second head 216. The head 216 is formed at the distal end of the second side arm 210, and has a second through-hole 217 that extends completely through the entire width of the head 216, transverse to the second longitudinal axis. The through-hole 217 can be a circle or oval shape, with an added notch at the top and/or bottom of the circle, to resemble a keyhole shape.

The head 216 also has a circular inward-facing face or surface 218. A second neck 214 is formed between the second main body section 212 and the second head 216. The second neck 214 is smaller than the main body section 212 to form a step-in or reduced portion that allows for material thickness of the pinch pad 220 silicone to be molded or placed over and about the head 216 and neck 214 (compare FIGS. 3A, 3B), so that the pinch pad 220 is flush with the inner and outer surfaces of the main section 212 at the neck 214. In addition, a rib can extend longitudinally at the reduced portion of the neck 214 from the main body section 212 to the head 216. The rib extends outward from the reduced portion to prevent rotation of the silicone. The main body section 212 extends from the curved corner 202 at the proximal end of the second side arm 210, to the neck 214.

As best shown in FIG. 3B, the main body section 212 has a rectangular cross-section. A channel 215 is formed at the inside of the main body section 212, which in turn forms side rails 213 that extend longitudinally along the forward-edge and rear-edge of the main body section 212, and to the rounded corner 202. The side rails 213 provide added support and strength to the main body section 212 and the rounded corner 202.

A second compression or pinch pad 220 is formed separately over and about the head 216, in the through-hole 217 and about the neck 214, to be coupled therewith. The through-hole 217 (including the notches), neck 214 and neck rib cooperate to enhance the coupling between the pinch pad 220 and the head 216 and neck 214, and to prevent rotation of the pinch pad 220 with respect to the second female external pressure member 200. Other coupling features can be added to further enhance coupling and prevent rotation of the pinch pad 220 with respect to the female external pressure member 200. The pinch pad 220 has a larger diameter than the second head 216, and has an inner surface that is cross-hatched to better grip the user's nose.

The second female engagement member 250 is a linear elongated tube. In some embodiments, the tube has an irregular cross-sectional transverse shape, for example, it can have symmetry in a radial direction, but lacks rotational symmetry, or is rotationally asymmetric. In the embodiment shown, the engagement member 250 has a rounded lower portion 252 and a flat top portion 254. The rounded lower portion 252 can be a circular arc and the top portion 254 is a chord connecting the endpoints of the lower portion 252, to resemble a U-shape with a top that is curved inward.

In the embodiment shown, the second engagement member 250 has a second engagement longitudinal axis, a distal end, and a proximal end. The female engagement member 250 is the same size and shape as the curved corner 202 (which is the same size as the second main body section 212), and a portion of the proximal end of the female engagement member 250 is received by the channel 215 of the curved corner 202, whereby the curved corner 202 is flush with the female engagement member 250. The female engagement member 250 is hollow, with a central opening extending the entire length of the engagement member 250.

As best shown in FIG. 3C, a sliding adjustment and lock mating feature in the form of an elongated slot or channel 270 is formed in the bottom of the lower portion 252 of the second female engagement member 250 and curved corner 202. The channel 270 extends the entire longitudinal length of the engagement member 250, from the proximal end at the curved corner 202, nearly to the opposite distal end of the engagement member 250. The channel 270 is spaced from the distal end to form a locking cross-member 272 that extends transversely at the distal end of the engagement member 250.

The width of the channel 270 is larger than the width of the locking tab 164, though the tongue 160 can be wider or narrower than the channel 270. Accordingly, the locking tab 164 can be freely and slidably received in the channel 270. In addition, the locking cross-member 272 has an inner edge 274 that engages with the lip 166 of the locking tab 164 of the male engagement member 150, to prevent the locking tab 164 from inadvertently escaping the channel 270 and decoupling the first arm 110 from the second arm 210.

### Slidable Adjustment of Engagement Members 150, 250 (FIGS. 4a-4c)

Comparing FIGS. 2A, 3A, the female engagement member 250 has the same cross-sectional shape as the male engagement member 150, but is slightly larger than the male engagement member 150. The female engagement member 250 has an inner circumference or perimeter that is larger than the outer circumference or perimeter of the male engagement member 150. That configuration allows the male engagement member 250 to be longitudinally, linearly, and slidably received in the central opening of the female engagement member 150, whereby the flat top 254 of the female member 250 aligns with the flat top 154 of the male member 150, and the rounded lower portion 252 of the female member 250 aligns with the rounded lower portion 152 of the male member 150. When the female engagement member 250 is removably and slidably coupled with the male engagement member 150, the engagement members 150, 250 are positioned between the two arms 110, 210.

Referring to FIGS. 4A-4C, to slidable couple the male external pressure member 100 to the female external pressure member 100, the male engagement member 150 is properly aligned with the female engagement member 250. The user then presses the locking tab 164 inward, so that the tongue 160 flexes inward with respect to the male engagement member 150 since the tongue 160 can flex inward at the slots 162. In some embodiments, the distal end of the locking tab 164 can be inward, so that the tab 164 need not be depressed. The distal end of the male engagement member 150 is then inserted into the distal end of the female engagement member 250. The locking tab 164 rides along the inner surface of the cross-support member 272. The user continues to press the engagement members 150, 250 together until the rear lip 166 of the locking tab 164 passes the inner edge 274 of the cross-support member 272. At that point, the bias of the tongue 160 pushes the tongue 160 outward, so that the locking tab 164 enters the female channel 270. The locking tab 164 remains in the female channel 270, and the rear lip 166 and inner edge 274 prevent the locking tab 164 from inadvertently being removed from the channel 270.

Referring to FIGS. 4A-4C, the engagement members 150, 250 have a fully-extended position (FIGS. 4A, 4B) and a fully-retracted position (FIG. 4C). The male engagement member 150 and first arm 110 linearly slides inward and outward with respect to the female engagement member 250 and second arm 210, respectively, between the fully-extended position and the fully-retracted position. The locking tab 164 remains in the channel 270 during that entire sliding motion.

In the fully-extended position (FIGS. 4A, 4B), the side arms 110, 210 are furthest apart from each other. The rear lip 166 of the locking tab 164 touches the inner edge 274 of the channel 270, which prevents the engagement members 150, 250 from inadvertently separating from each other. In the fully-extended position, the arms 110, 210 are wider than a person's nose and can be aligned with the side of a person's nose.

In the full-retracted position (FIG. 4C), the arms 110, 210 are moved fully inward toward each other. The locking tab 164 is at or near the distal end of the channel 270 and female engagement member 250.

Due to the flat tops 154, 254, the engagement members 150, 250 are rotationally asymmetric, which prevents the male engagement member 150 from rotating with respect to the female engagement member 250. Accordingly, the first side arm 110 and first head 116 remain aligned with the second side arm 210 and second head 216, respectively, as the user moves the engagement members 150, 250 together or apart. It is further noted that the cross-section of the engagement members can be axially symmetric and have at least one angle or bend, such as a square, rectangle, or triangle, so that the engagement members are rotationally asymmetric. However, an irregular shape means that the engagement members can only be inserted in a single alignment that properly aligns the arms 110, 210; whereas a square or rectangle can be inserted but be in a wrong alignment of the arms 110, 210. In addition, the engagement members can have a circle cross-section that allows rotational movement, but rotation is prevented by the lock tab 164 in the channel 115.

In addition, referring to FIG. 1B, one or more grip features 104, 204 are provided on the outer surface of the device 10. In the illustrated example embodiment, the grip feature 104, 204 is shown as a thin elongated projection that extends outward from the top surface of the corner 102, 202. In some embodiments, the grip features 104, 204 can be located elsewhere on the device 10, such as on the main body section 112, 212 of the arm 110, 210. The grip feature 104, 204 has a bend that defines a first leg portion and a second leg portion. The first leg portion extends vertically (in the embodiment of FIG. 1B), orthogonal to the longitudinal axis of the engagement member 150, 250, and the second leg portion extends approximately 45 degrees outward from the first leg portion along the curved corner 102, 202. The grip 104, 204 is located on both sides of the external pressure member 100, 200.

The grips 104, 204 provide an added surface for the user to better grip the external pressure member 100, 200 during placement about the user's nose, and to slidably push the arms 110, 210 inward and retract the arms 110, 210 outward. The user can further push the arms 110, 210 inward by pressing on the outer surface of the arms 110, 210 or the corners 102, 202.

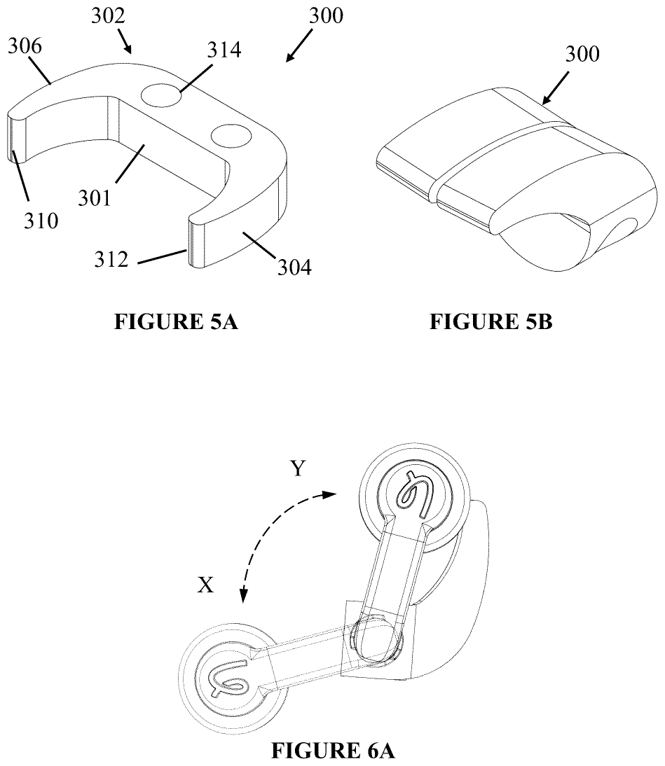

### Internal Member 300 (FIGS. 5a, 5b)

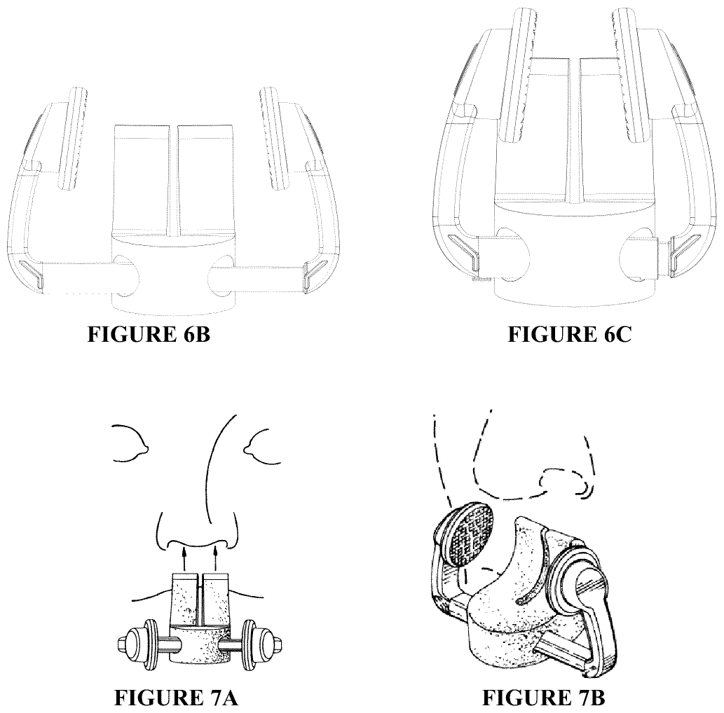

FIGS. 5A, 5B show the internal member 300 in greater detail. The internal member 300 attaches to the male and female engagement members 150, 250. The internal member 300 has a body 301 with a central portion 302 and two opposing arms 304, 306. The internal member 300 has a general U-shape, with the arms 304, 306 extending in the same direction forward from the central portion 302. Referring to FIGS. 1C, 5B, the arms 304, 306 are elongated with a curved outer surface and a curved inner surface. The arms 304, 306 have a wider proximal end at the central portion 302 and have a curved taper outward to a narrower distal end with a distal tip 310, 312, respectively. The arms 304, 306 extend at about 20-60 degrees to the central portion 302, and in one embodiment about 30 degrees to the central portion 302, as best shown in FIG. 1C. And, as shown in FIG. 6A, the internal member arms 304, 306 are slightly shorter than the device arms 110, 210, though in other embodiments the internal member arms 304, 306 can be shorter, longer or the same length as the device arms 110, 210.

Referring to FIG. 5A, one or more openings 314 are located in the central portion 301 that extends completely through the width of the central portion 302.

The first and second arms 304, 306 of the internal member 300 are each configured to be shaped and sized to fit in a person's nose. Each arm 304, 306 is inserted into each nare before the external pressure members 100, 200 are pinched in place. Once the internal member 300 is inserted into the nasal passage, they compress flat when pinched which is more comfortable in the nose. In some embodiments, the internal member 300 (or at least the distal ends that extend inside the nose) can be saturated with a medication prior to insertion. When the internal member 300 is squeezed, it compresses to release medication or absorbs and provides some internal tamponade. For example, the internal member 300 can release the medication in the nasal mucosa so that liquid comes in contact with areas of bleeding. Thus, the internal member 300 can be used without medication to stop a nosebleed, and can be used with medication as a medicinal applicator either to further stop a nosebleed or to apply a medication for other purposes.

In one exemplary embodiment, the internal member 300 may be a nasal sponge and can be made for instance of foam, sponge, dehydrated sponge like materials, and can optionally be presoaked with medication (and optionally dehydrated). The internal member 300 can be biocompatible foam that absorbs blood and/or induces clot formation or hemostasis. The primary purpose of the sponge is to deliver medication, but the sponge can also provide an expanding absorbent material that provides internal tamponade, or can be configured to exert a pressure against the nose to stop bleeding. In addition, the nasal sponges 300 may be made out of absorbable polymers, sponges or other material that can be combined or coated with zinc oxide, bacitracin or antibiotic ointment in addition to analgesic and vasocontrictive medications such as but not limited to oxymetazoline, epinephrine, phenylephrine, pseudoephedrine, lidocaine or tranexamic acid (TXA).

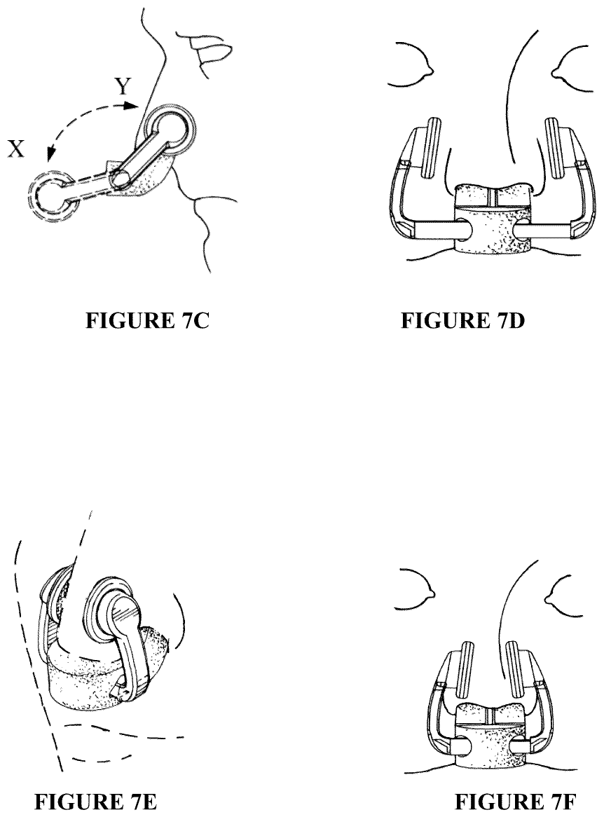

As shown in FIGS. 7A-7F, the arms 304, 306 of the internal member 300 extend upward upon entry and then posterior into the nare, while the base of the arms 304, 306 may project slightly out of the nasal passage at the exterior of the nose. The nasal sponge arms 304, 306 fit securely below the nose but above the level of the upper lip to be comfortably positioned in the nasal passages, and can be readily inserted into and withdrawn from the nasal passages. The sponge arms 304, 306 can easily slide into the nare and quickly and easily release pre-soaked medication to mucous membranes inside the nasal shaft. They do not adhere to or disrupt scab formation. The sponge 300 and sponge arms 304, 306 can be provided in different sizes, shapes and angles to fit different ages (nose size) and internal nose shapes.

The entire internal member 300 is formed as a single unitary piece, including the central portion 302 and the arms 304, 306. This provides better placement within the nasal passage, and easier removal from the nasal passage after use since the central portion 302 assists with removal of the sponge arms 304, 306. In addition, the one piece ensures that the internal member 300 doesn't detach and become lodged/stuck in the nare or nasal passage. In one embodiment, the internal member 300 is a one-piece sponge. However, in other embodiments, the internal member 300 can be two or more pieces that are separately held in position by separate support or attachment members such as a fastener or adhesive that are made of a different material than the internal member 300, such as plastic or metal, and can be rigid or flexible. In some embodiments, the top and bottom surfaces (which forms the bottom and top surfaces, respectively, when the arms are folded together, as in FIG. 5C) of the arms 304, 306 are curved, though in other embodiments, the top and bottom surfaces are straight.

### Assembly

The first male external pressure member 100, second female external pressure member 200, and the internal member 300 are discrete and separate from one another. To assemble the device 10, the internal member 300 is initially positioned on the engagement members 150, 250. To do so, the arms 304, 306 of the internal member 300 are folded up (compare FIG. 5A and FIG. 5B) so that the arms 304, 306 to come together with both of the arms 304, 306 facing in the same direction. The two openings 314 are aligned with each other. One or both of the engagement members 150, 250 are then pushed into the holes 314. The male engagement member 150 is aligned with and slidably coupled to the female engagement member 250, as described above. Once the sponge 300 is in position and the engagement members 150, 250 coupled together, the sponge 300 cannot be removed without decoupling the engagement members 150, 250. The openings 314 are larger than the engagement members 150, 250 so that the engagement members 150, 250 (and arms 110, 210) can freely rotate in the openings 314 with respect to the sponge arms 304, 306. That facilitates the ability of the user to position the sponge arms 304, 306 in the user's nose without the engagement members 150, 250 impeding the rotation.

At this point, the internal member 300 is reliably attached to the engagement members 150, 250, and cannot come free during normal use of the device 10. Medication can optionally be placed on the internal members 300, and sealed within a bag to prevent the medication from escaping or evaporating. The external pressure members 100, 200 can optionally be coated in plastic so that it does not rust or chemically interact or interfere with the medication, for instance a skin-safe plastic dip. The pressure members 100, 200 can be made of a rigid plastic or material so that it does not chemically interact or interfere with the medication, and the pinch pads 120, 220 can made of rubber to better grip the user's nose.

The device is now fully assembled and ready for use. In some embodiments, the device 10 is fully preassembled so that the user (e.g., medical practitioner or patient) need not assemble the device 10. In addition, the device 10 can be disposable after a single use. In some embodiments, the device 10 can be reused by removing and discarding the internal member 300 after use, cleaning the support members 100, 200, and placing a new internal member 300. In other embodiments, the internal member 300 can be cleaned and reused by being re-placed onto the support members 100, 200. The sponges can be checked after 10-20 minutes, rinsed with clean water and reinserted for a total of 30 minutes, though other times can be utilized. The male engagement member 250 is removed from the female engagement member by depressing the locking tab 164 inward until it can pass the cross-support member 272, and then by pulling the engagement members 150, 250 apart. The internal member 300 can then be removed from the engagement members 150, 250 and replaced or reused.

### Overall Operation (FIGS. 6, 7)

Operation of the device 10 will be discussed with respect to FIGS. 6A-6C and 7A-7F. The engagement members 150, 250 of the device 10 are moved to the fully extended position (FIG. 6B). If needed, the user can move the arms 110, 210 outwardly with respect to each other, in the direction of the arrow X (FIG. 6A), to provide an appropriate separation. Thus, the pinch pads 120, 220 are wider apart than the user's nose so that the user can insert the nasal sponge arms 304, 306 into the user's nasal passages by moving the pinch pads 120, 220 upward (and posteriorly) along the external lateral side surface of the user's nose.

Referring to FIG. 6A, the device is placed in an insertion position to prepare the device for insertion into the user's nose. If needed, the user rotates the arms 110, 210 with respect to the arms 304, 306 of the internal members 300, and away from the arms 304, 306 of the internal members 300, in the direction of the arrow X as shown. This moves the arms 110, 210 and pinch pads 120, 220 away from the user and the distal ends 310, 312 of the sponge arms 304, 306.

Accordingly, the sponge arms 304, 306, and particularly the distal ends 310, 312, are free of the arms 110, 210 and the pinch pads 120, 220, so that the arms 110, 210 do not interfere with insertion of the sponge arms 304, 306 into the nasal passage of the user.

Turning to FIGS. 7A, 7B, the user then inserts the distal ends 310, 312 of the sponge arms 304, 306 into the user's nasal passage in the direction of the arrows shown. In FIGS. 7C, 7D, the sponge arms 304, 306 are inside the user's nasal passage, and apply an outward pressure to stop bleeding. At this point, the arms 110, 210 and the pinch pads 120, 220 are facing away from the user, as shown. The user then moves the device 10 into a pre-pinch position. The user rotates the arms 110, 210 with respect to the arms 304, 306 of the internal members 300 and toward the arms 304, 306 of the internal members 300 as shown by the arrow Y (FIG. 6A), so that the pinch pads 120, 220 come up alongside the outside of the user's nasal passage, as shown in FIGS. 7C-7F.

In FIGS. 7D-7F, the user places the device 10 in the closed position. The user presses inward on the arms 110, 210 or the pinch pads 120, 220, until the pinch pads 120, 220 engage the user's nasal passage. In the pinch position, the arms 110, 120 are positioned inward so that the distal end portion pushes inward on the user's nose. The pinch pads 120, 220 apply an inward pressure to help stop the nosebleed. The pinch pads 120, 220 will remain in that position and apply constant pressure for up to 30 minutes or more, until bleeding has stopped, and the user need not manually hold the device 10 in position.

It is further noted that if no pressure is applied to the arms 110, 210, the engagement members 150, 250 can freely slide inward and outward with respect to one another. However, the first and second engagement members are directionally locked in position and cannot move outward with respect to each other, when an outward force applied to said first and second compression pads. That outward force applies a radial force to the first and second engagement members.

Thus, when a radial force is applied is applied, such as when the pinch pads 120, 220 are pressed against the user's nose, that radial force moves the first engagement member 110 transversely against the second engagement member 210. As shown in FIG. 4B, for example, the distal end of the male engagement member 150 moves upward in the direction A with respect to the female engagement member 250, and the distal end of the female engagement member 150 moves downward in the direction B with respect to the male engagement member 150. That radial force creates a pressure lock between the engagement members 150, 250, until released by the user. In addition, the device 10 is made of a rigid plastic, but is relatively thin so that the arms 110, 210 and engagement members 150, 250 can flex slightly, which enhances the pressure lock. In addition, it is noted that the arms 110, 210 and engagement members 150, 250 cannot be separated by pulling outward on the pinch pads 120, 220, since that increases the radial force, but rather should be separated by pulling outward on the bottom (proximal) end of the arms 110, 210, for example at the grip features 104, 204, which does not apply a radial force.

However, the arms 110, 210 are in a fixed position relative to the respective engagement member 150, 250, where the arms 110, 210 are approximately orthogonal to the respective engagement member 150, 250, or slightly less than orthogonal (80-90 degrees) to bend inward so that the pressure pads 120, 220 can touch the user's nose without obstruction by the arms 110, 210, forming a general L-shape or curved L-shape.

Thus, the arms 110, 210 remain in that pinched position until separated by the user for removal from the nose by separating the arms 110, 210 outwardly, including to release the radial force by rotating the engagement arms in an opposite direction upward/downward with respect to each other and opposite to the radial force. In some embodiments, the user can pull the device 10 downward off of the nose, to release the radial force.

FIG. 4B also illustrates that the pinch pads 120, 220 have inner planar surfaces. Those inner surfaces are not parallel to each other, but rather expand outwardly from the distal end of the arms 110, 210 toward the proximal end of the arms 110, 210. That tapered configuration accounts for the shape of the nose, which is wider at the bottom.

In the insertion position and pre-grip position (FIGS. 7C, 7D) and grip position (FIGS. 7E, 7F), the engagement members 150, 250 are positioned at the patient's upper lip directly below the nostrils and septum. The device arms 110, 210 extend upward on the patient along the lateral surface of the nose. The distal ends are positioned at the lateral surface of the nose, so that the external pads 120, 220 apply pressure at a proper position to stop the nosebleed. The arms 110, 210 exert an inward force so that the pinch pads 120, 220 apply an inward pressure or pinch force against the lateral surface of the nose, while at the same time the sponge arms 304, 306 are positioned inside the nose and apply an outward force. The nasal pads 120, 220 thereby apply pressure to the ala (soft side walls of the nose). The pinch force can be controlled by adjusting the amount of inward pressure applied by the arms 110, 210, so that enough force is applied to control the bleeding, but without exerting too much force to cause pain or damage the user's nose.

As shown in FIGS. 7E, 7F, once the device 10 is positioned on the nose, it is hands-free since it remains in place on the user's nose by itself due to the inward pinch force once the arms 110, 210 are moved to the inward pinch position (which need not be the fully-retracted position), and without the user having to hold it in position. The device 10 remains in position until the user separates the arms 110, 210 and then withdraws the nasal sponge 300 from the user's nasal passage. Thus, the device 10 (i.e., distal end portions and pads 120, 220) pinches the lateral side surfaces of the user's nose, and simultaneously stops bleeding through delivery of vasoconstrictive medications applied by the nasal sponge 300. Of course, the device 10 need not have both the pinch pads 120, 220 and the nasal sponge 300. For instance, the sponge 300 can be removed, so that only a pinching force is applied by the distal end portion (head 116) and pads 120, 220.

The vast majority of nosebleeds occur in the anterior part of the nose and from the nasal septum. This area contains many blood vessels from Kiesselbach's plexus, also known as Little's area. The nasal compression device 10 is designed to control bleeding via direct compression on the outer nose, by placing pressure directly over Kiesselbach's plexus, in combination with vasoconstriction from sponge inserts soaked with oxymetalozine or other vasocontricting medication or hemostatic agent possible with some analgesic ingredient such as lidocaine and/or antibacterial agent such as bacitracin. The device 10 may also be designed to come in multiple sizes to fit adults and children.

The device can be made available in several sizes, such as child, teen, and adult. Each size may have, for instance, a different width (e.g., by varying the length of the central portion 102), a different height (e.g., by varying the length of the arms 110, 210). The different sizes can also have different sizes of nasal pads 120, 220 and nasal sponge arms 304, 306.

### Conclusion

The nose compression device has two L-shaped or curved L-shaped support members, each having elongated arms with a compression pad formed at a distal end, and an elongated engagement member at the proximal end. The engagement members slidably couple with one another and couple with sponge(s) that are inserted into a patient's nose. To apply the device, the engagement members are moved inward until the compression pads press against a user's nose. The outward force created by the user's nose pressing outward against the compression pads, creates a radial force at the engagement members that allows the device to be further pressed inward, but prevents the engagement members from being moving inward, thereby locking the device to the user's nose. A releasing outward force can be applied along the engagement members to release the radial force and move the engagement members outward so the device can be removed from the user's nose. Releasable locking features are formed on the engagement members to prevent the engagement members from inadvertently coming apart. However, a user can unlock the locking features to remove and replace or reuse the sponges.

The device 10 provides a safe, effective and low-cost solution to nosebleed emergencies. The device comes in adjustable sizes and applies constant hands-free pressure to the soft side walls of the nose, incorporates intranasal sponges to control bleeding, and is designed to apply a constant adequate pressure to the nose for the appropriate amount of time (e.g., 10-20 minutes). The device can also utilize medication that further controls bleeding by constricting bleeding vessels and improves the success of hemorrhage control.

In some embodiments, the device 10 need not be used to compress the nose to stop a nosebleed, but instead can be utilized for other purposes and applications, for example, to deliver medications by placing the medication on the arms 304, 306 of the internal member 300, such as analgesic, allergy medications, benzodiazepines, naloxone, angelistic, migraine medications, vaccines or other medications that can be applied intranasally, etc. This is especially useful in an emergency or other situations where IV access or taking pills cannot be well tolerated, such as for conditions other than nosebleeds. In those embodiments, the arms 304, 306 and pinch pads 120, 220 need not be positioned to compress the nose to stop a nosebleed, but apply a lighter pressure to remain in place as the medication is delivered via the arms 304, 306 of the internal member 300.

The present device 10 provides value to three different customer segments: medical providers, sports medicine professionals and parents, caretakers or individuals who suffer from frequent nosebleeds. For medical providers, this device can reduce the time needed by medical professionals to effectively manage common nosebleeds. For coaches, trainers and school nurses, this device is easy to use and effective for nosebleed emergencies. For parents and caretakers, the device is the comfortable, safe, low-cost solution to nosebleed rescue that can reduce or eliminate costly visits to the emergency room or doctor's office.

The device is available in adjustable sizes and applies constant hands-free pressure to the soft side walls of the nose, incorporates cooling to help constrict vessels to control bleeding, and is designed to automatically apply adequate pressure to the nose for 10-20 minutes. The device also uses medication that further controls bleeding by vasoconstriction and improves the success of hemorrhage control.

The device is a safe, effective and simple to use device that can be placed on the patient by a nurse, an emergency room clinician or family member and does not require constant supervision by a medical professional. It allows providers to effectively manage epistaxis with minimal cost, time or repeated attempts and helps to facilitate throughput which reduces length of stay, an important hospital benchmark. These advantages make the present device attractive not only to the beneficiary, the patient, but also to the hospital or physician who recommends use of the product.

The device is easy to use, stops bleeding immediately and has step-by-step instructions for managing nosebleeds. It is a device that any trainer or first aid station would want to have on hand for disposable, immediate and effective treatment for nosebleed emergencies. Any customers who are prone to nosebleeds would be attracted to the low price, intuitive design, effectiveness and comfort. The device provides easy step-by-step instructions to apply constant pressure to the correct part of the nose with the added value of including medication which increases the success of bleeding control and potentially prevents unnecessary visits to the doctor's office or emergency room. Currently, there are no devices on the market that use this combination of technique and medication to treat nosebleed emergencies. The device is valuable because it provides cost savings, effective management and ease of use to (1) medical providers/physicians, (2) school and sports professionals and (3) direct to consumer customer segments and by preventing prolonged nosebleeds it avoids overutilization and expense to the health care system.

The device 10 is adjustable for comfortable fit to the contour of the nose, since the nasal pads 120, 220 are soft and malleable and with applied pressure conforms to the shape of the nose. As set by the user, it applies the appropriate amount of pressure in the appropriate location at the soft side walls of the nose, hands-free. It is positioned completely below or to the side of the nose, and does not extend upward above the soft side walls of the nose. Thus, it does not obstruct vision or line of sight of the user's eyes. It also does not extend below the upper lip, so that it does not obstruct the user's mouth and can be used with a surgical mask.

It is noted that the device 10 as shown and described is configured for simultaneous use on both nostrils. However, the device 10 can be configured so that only one nostril is treated, such as by providing one internal member arm. In addition, the device can be used without the internal member 300; for example, the pinch pads 120, 220 can be used by itself. The device 10 is designed for hands-free application of a constant pressure that the person experiencing the nosebleed can apply alone without the assistance of another person or a medical technician. In addition, while the device 10 has been described for use to stop nosebleeds, it can be used for other purposes; for example, the device can be a medication applicator that administers a medication to a patient and the pinch pads optionally provides only sufficient pressure on the nose to keep the device positioned on the nose.

In one embodiment, the nasal sponge is one piece and die cut, the compression members 100, 200 are injection molded, and has a silicone overmold. The components include the medical grade open cell intranasal sponge that can have a liquid or gel medication added to, the sponge is one piece that slips over the engagement members 150, 250 and allows for rotation of the sponge. The arms 110, 210 can be pulled apart and then pinched shut to hold pressure per user's needs and the silicone (or other plastic) pinch pads that provide compressible but comfortable pressure over the nostrils preventing any leakage of air or fluid. It will further be appreciated that any suitable materials can be used for the device, other than those disclosed, such as for the clip wire or plastic/silicone pinch pads, and provide constant pressure on the nostrils. In addition, other structures can be utilized to provide constant pressure on the nostrils within the spirit and scope of the disclosure.

The device 10 is designed to exploit the advantageous anatomical features of Kiesselbach's plexus. Located in the anterior part of the nasal septum, Kiesselbach's plexus stands out as a highly vascularized area, receiving blood supply from both the internal carotid artery (ICA) and the external carotid artery (ECA). This unique vascular structure makes it an ideal site for effective drug absorption directly into the systemic circulation. The strategic use of Kiesselbach's plexus by the device 10 for drug delivery is particularly advantageous for several reasons. Firstly, the area's rich vascularization ensures rapid absorption and onset of medication action, a critical factor in emergency medical situations or for managing chronic conditions that require consistent medication levels. This direct access bypasses the gastrointestinal tract, eliminating concerns related to oral drug administration, such as first-pass metabolism and digestive tract irritation. Secondly, the anterior location of Kiesselbach's plexus makes it easily accessible, allowing for the non-invasive application of the device. This is especially beneficial for patients who may experience anxiety or discomfort with other routes of administration, such as injections. The non-invasive nature of this delivery system is particularly appealing for pediatric patients or individuals with conditions that contraindicate invasive procedures, providing a stress-free method for both acute and chronic medication management. Moreover, the ability of the device 10 to deliver drugs through Kiesselbach's plexus capitalizes on the physiological response of the nasal mucosa, promoting efficient and immediate drug absorption. This is crucial for delivering a wide range of therapeutics, from hemostatic agents required for managing acute hemorrhagic conditions to essential medications like antidotes, antibiotics, analgesics, and anti-anxiety treatments.

The device employs TN730, 30A Durometer Silicone for the overmold, featuring a textured, non-slip surface and adjustable dimensions of the nasal pinch pads and base thickness to suit a wide array of nose types and sizes, thereby broadening its applicability. The intranasal foam component underwent a critical material shift from Polyurethane Foam to Woodbridge INOAC Hydrophilic Polyurethane Foam to eliminate any unpleasant odors and prevent drying, ensuring the foam's consistent softness and compressibility. This medical-grade, open-cell foam is designed to precisely absorb and retain the required volume of liquid medication, facilitating effective and comfortable drug delivery.

The device represents a significant advancement in the field of intranasal drug delivery, leveraging pharmacologic and dynamic considerations to offer a compelling value proposition. The intranasal route, recognized for its rich vascularization, offers direct access to the central nervous system (CNS), providing a non-invasive alternative with potential for bypassing the blood-brain barrier and achieving rapid onset of action akin to intravenous (IV) administrations. Traditional intranasal applications, predominantly in the form of sprays and aerosols, face challenges such as limited retention time on the mucosal surface and variability in administration techniques, which can compromise the effectiveness of the medication delivered.

The device 10 distinguishes itself by applying stable pressure to key anatomical absorption regions within the nasal cavity, thereby addressing two critical limitations of conventional intranasal delivery methods: (1) enhancing the retention time of drugs with poor absorption profiles and (2) potentially facilitating faster drug absorption for more rapid systemic delivery. This innovative approach could potentially improve the bioavailability of medications, including those not traditionally suited for nasal spray application due to their absorption characteristics.

The value proposition of the device 10 is further underscored when considering the user experience with existing intranasal delivery devices. Instructions for devices like nasal sprays and atomizers can be complex and challenging for untrained users, requiring specific handling and administration techniques. In contrast, the device 10 simplifies this process by eliminating the need for manual nasal compression, thereby offering a more user-friendly and efficient delivery method. This advantage aligns with the device's design to enhance usability and patient compliance, particularly in emergency situations where quick and effective medication administration is crucial. The significance of these features has been validated through preliminary research and feedback from healthcare professionals, indicating a strong market need for more efficient and user-friendly intranasal drug delivery solutions. The device 10 is supported by its unique benefits, including improved drug efficacy, ease of use, and the potential to reduce the need for hospital visits for conditions that can be effectively managed with intranasal medication.

The device's design eliminates the need for components like metal wires, to reduce injury, manufacturing complexity and cost. In addition, the device is more lightweight and better functions with a firm hold on the nose that can still be adjusted. The device uses silicone pinch pads that hold pressure on the nose but the arms of the device use interlocking plastic arms that slide into place. The arms 110, 210 and engagement members 150, 250 are injection molded as a single unitary piece of plastic (no metal), and the pinch pad 120, 220 is a silicon overmold held in place by friction and the coupling features at the neck (reduced diameter with steps) and/or head (through-hole 117). Because there is no plastic, the device is lightweight, but the rigid plastic is durable.

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

It is further noted that the drawings may illustrate and the description and claims may use several geometric or relational terms and directional or positioning terms, such as planar, curved, elongated, circular, rounded, tapered, stepped, parallel, perpendicular, orthogonal, transverse, axially rectangular, circular, flat, vertical, horizontal, top, bottom, left, right, up, down, inner, outer, side, distal, and proximal. Those terms are merely for convenience to facilitate the description based on the embodiments shown in the figures, and are not intended to limit the disclosure. Thus, it should be recognized that the disclosure can be described in other ways without those geometric, relational, directional or positioning terms. In addition, the geometric or relational terms may not be exact. For instance, walls or surfaces may not be exactly flat, perpendicular or parallel to one another but still be considered to be substantially perpendicular or parallel because of, for example, roughness of surfaces, tolerances allowed in manufacturing, etc. And, other suitable geometries and relationships can be provided without departing from the spirit and scope of the disclosure.

Within this specification, the various sizes, shapes and dimensions are approximate and exemplary to illustrate the scope of the disclosure and are not limiting. The sizes and the terms “substantially” and “about” mean plus or minus 15-20%, or in other embodiments plus or minus 10%, and in other embodiments plus or minus 5%, and plus or minus 1-2%. In addition, while specific dimensions, sizes and shapes may be provided in certain embodiments of the invention, those are simply to illustrate the scope of the disclosure and are not limiting. Thus, other dimensions, sizes and/or shapes can be utilized without departing from the spirit and scope of the disclosure.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfill the requirements of uniqueness, utility, and non-obviousness.

In addition, the statements made with respect to one embodiment apply to the other embodiments, unless otherwise specifically noted. It is further understood that the description and scope of disclosure apply equally (though the descriptions have not been repeated) for each structure that is the same or similar between each of the various embodiment, and whether or not those structures have been assigned a similar reference numeral.

Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting.

1. A nose compression device for use on a person, the device comprising:
a first elongated arm having a first distal end and a first proximal end;
a first compression pad formed at the first distal end of said first arm;
a first elongated engagement member at the first proximal end of said first arm, said first engagement member having a first coupling feature;
a second elongated arm having a second distal end and a second proximal end;
a second compression pad formed at the second distal end of said second arm;
a second elongated engagement member at the second proximal end of said first arm, said second engagement member having a second coupling feature configured to slidably couple with said first coupling feature.

2. The nose compression device of claim 1, wherein said first engagement member is releasably engaged with said second engagement member.

3. The nose compression device of claim 2, further comprising an absorbent internal member removably coupled to said nose compression device, wherein said internal member can be removed and replaced.

4. The nose compression device of claim 3, wherein said internal member can be removed and replaced when said first engagement member is disengaged from said second engagement member, and said internal member is locked to said nose compression device when said first engagement member is engaged with said second engagement member.

5. The nose compression device of claim 1, wherein said first engagement member comprises a first hollow tube having a lower portion and a distal end, and said first coupling feature comprises a tongue formed in the lower portion of said first engagement member, said tongue defining two parallel slots formed in the lower portion of said first engagement member, and a locking tab at the distal end of said first engagement member;
wherein said second engagement member comprises a second hollow tube having a lower portion and a distal end, and said second coupling feature comprises an elongated channel formed in the lower portion of said second engagement member and a cross-support member formed at the distal end of said second engagement member;
wherein said locking tab enters the elongated channel and releasably locks said first engagement member to said second engagement member.

6. The nose compression device of claim 1, further comprising an elongated sponge rotatably coupled to said first and second engagement members, said sponge extending outward from said first and second engagement members and configured for insertion into the person's nasal passage.

7. The nose compression device of claim 6, wherein said sponge contains a medication.

8. (canceled)

9. The nose compression device of claim 6, wherein said sponge can be removed from said nose compression device and replaced for reuse.

10. The nose compression device of claim 6, said nose compression device having an insertion position whereby said first and second arms are rotated with respect to said elongated sponge to face away from the user as the user inserts the elongated sponge into the person's nasal passages; and
said nose compression device having a postinsertion position whereby the first and second arms are rotated with respect to said sponge to align along a side of the person's nasal passages; and
said nose compression device having a pinch position whereby the first and second arms are pressed inwardly toward each other to pinch the person's nasal passages.

11-13. (canceled)

14. The nose compression device of claim 1, wherein each of said first and second arms have a distal end, and further comprising a pinch pad molded to the distal end.

15. (canceled)

16. The nose compression device of claim 1:
said first arm comprising a first head at the first distal, a first neck coupled with the first head, and a first main body section coupled with the first neck, said first neck having a first neck coupling feature, and said first head having a first head coupling feature, wherein said first compression pad is formed over said first head and said first neck, and said first neck coupling feature and said first head coupling feature enhance coupling of said first compression pad and prevents rotation of said first compression pad; and
said second arm comprising a second head at the second distal, a second neck coupled with the second head, and a second main body section coupled with the second neck, said second neck having a second neck coupling feature, and said second head having a second head coupling feature, wherein said second compression pad is formed over said second head and said second neck, and said second neck coupling feature and said second head coupling feature enhance coupling of said second compression pad and prevents rotation of said second compression pad.

17. (canceled)

18. The nose compression device of claim 1:
said first engagement member comprises a first hollow tube with at least one first bend; and
said second engagement member comprises a second hollow tube with at least one second bend;
wherein said at least one first and second bends prevent said first engagement member from rotating with respect to said second engagement member.

19. (canceled)

20. The nose compression device of claim 1, wherein said first and second engagement members are directionally locked in position and cannot move outward with respect to each other, by an outward force applied to said first and second compression pads, and wherein the outward force applies a radial force to said first and second engagement members.

21. (canceled)

22. The nose compression device of claim 20, wherein said first and second engagement members can be moved further inward by an inward force. and wherein said first and second engagement members can be moved outward by an outward force applied to said first and second engagement members.

23. (canceled)

24. The nose compression device of claim 22, further comprising:
a first bend connecting said first elongated arm with said first engagement member; and
a second bend connecting said second elongated arm with said second engagement member;
wherein said first and second engagement members can be moved outward by an outward force applied to said first and second bends.

25. The nose compression device of claim 24, further comprising:
a first grip located on said first bend;
a second grip located on said second bend;
wherein said first and second engagement members can be moved outward by an outward force applied to said first and second grips.

26. A nose compression device for use on a person, said device comprising:
a first elongated external pressure member comprising:
a first elongated side arm having a first main body section, first arm distal end and a first arm proximal end;
a first neck coupled with the first main body section;
a first head coupled with said first neck at the first arm distal end;
a first compression pad formed about said first head;
a first elongated linear engagement member coupled with said first elongated side arm, said first linear engagement member comprising a first tube that is rotationally asymmetric, and a first locking feature;
a second elongated external pressure member comprising:
a second elongated side arm having a second main body section, second arm distal end and a second arm proximal end;
a second neck coupled with the second main body section;
a second head coupled with said second neck at the second arm distal end;
a second compression pad formed about said second head;
a second elongated linear engagement member coupled with said second elongated side arm, said second linear engagement member comprising a second tube that is rotationally asymmetric, and a second locking feature;
wherein said first engagement member is slidably coupled with said second engagement member; and
wherein said first locking feature and said locking feature prevent said first engagement member from decoupling with said second engagement member.

27. The nose compression device of claim 26:
wherein said first tube is hollow and said first locking feature comprises a tongue at a first tube distal end of said first tube, and a locking tab having a ramp at the first tube distal end on said tongue;
wherein said second tube is hollow and said second locking feature comprises an elongated channel formed in a bottom of said second tube with a cross-section support member; and
wherein said locking tab is slidably received in said elongated channel, and said cross-section support member prevents said locking tab from inadvertently decoupling with said elongated channel.

28. The nose compression device of claim 26, wherein said first tube is hollow and said second tube is hollow.

29. The nose compression device of claim 26, wherein said first and second engagement members are directionally locked in position and cannot move outward with respect to each other, by an outward force applied to said first and second compression pads.
